(19) United States
(12) Patent Application Publication
(10) Pub. No.: US 2015/0352304 A1
(43) Pub. Date: Dec. 10, 2015

(54) Bite Proof Endotracheal Tube

(71) Applicant: Gemguardian, LLC, Fox Point, WI (US)

(72) Inventor: Yakira Leevan, Fox Point, WI (US)

(21) Appl. No.: 14/792,233

(22) Filed: Jul. 6, 2015

Related U.S. Application Data
(60) Provisional application No. 62/131,453, filed on Mar. 11, 2015.

(51) Int. Cl. A61M 16/04; A61M 16/14; A61M 16/08; A61M 16/00

(52) U.S. Cl. CPC A61M 16/0493; A61M 16/0057; A61M 16/044; A61M 16/14; A61M 16/0875

(57) ABSTRACT

An endotracheal tube assembly that is constructed to prevent kinking and crushing of the tube due to interaction with the dontia of a patient or a patient position, orientation, or movement. The endotracheal tube assembly includes a tube defined by a hollow core that extends between a first end and a second end of the endotracheal tube assembly. A reinforcement is formed integrally with the endotracheal tube assembly and constructed to prevent constriction of the hollow core.

### CROSS-REFERENCE TO RELATED APPLICATIONS

This application claims priority to U.S. Provisional Patent Application Ser. No. 62/131,453 filed on Mar. 11, 2015 titled “Bite Proof Endotracheal Tube” and the disclosure of which is incorporated herein.

### BACKGROUND OF THE INVENTION

The present invention is directed to an endotracheal tube assembly that is constructed to mitigate or otherwise reduce or wholly avoid instances of kinking and/or patient compression of the tube and which is usable in various patient and assembly positions.

An endotracheal tube is a flexible plastic tube inserted through the mouth of the patient and into the trachea to maintain an unobstructed passageway to deliver respiration gases such as air, oxygenated air, oxygen, and/or inhalation anesthetics to the lungs of a patient. Patients are commonly unconscious during intubation but some circumstances may require that the patient maintain consciousness during intubation.

In adult patients the endotracheal tube is commonly placed at a tube depth of approximately 20-23 cm as determined by a distance between an in-situ end of the tube and the upper incisor teeth of a patient. Commonly, the tube insertion depth is associated with a patient gender, age, and/or anatomy to achieve the desired introduction of respiration gases to the lungs of a patient rather than other anatomy. An inflatable bladder or cuff is commonly associated with an insertion end of the endotracheal tube and, when inflated, maintains a desired position of the endotracheal tube relative to the patient. The inflatable cuff also mitigates or prevents directing the intubation gases to other anatomy, such as the stomach, of a patient.

A first end of the endotracheal tube remains exposed to atmosphere via the mouth of the patient and facilitates the ability of a care provider to communicate desired respiration gases and/or medicinals to the lungs of a patient. The first end of the endotracheal tube also facilitates the doctor or technicians ability to interact with and control the configuration of the inflatable bladder or cuff during insertion, placement, and removal of the endotracheal tube. As the intubation tube assembly is the sole means of patient respiration, particular attention must be given to the placement and desired operation of the endotracheal tube assembly to maintain the desired patient intubation and/or respiration as any disruption or constriction of the passage associated with the endotracheal tube assembly, even for a very limited duration, can have dire consequences to the health of an intubated patient.

The wall thickness of the endotracheal tube assembly is another concern that must be considered when providing an endotracheal tube assembly. For instance, pediatric endotracheal tubes have thinner wall dimensions than endotracheal tubes provided for other ages of users. These thinner walled pediatric endotracheal tube assemblies are likely to be rendered wholly inoperative or Impassable if kinked. Kinking of such endotracheal tube assemblies can be disastrous if unnoticed or in case of delayed attendance during patient management.

Regardless of patient size, intubation is generally considered an uncomfortable if not painful experience for many users. Patients who regain consciousness while intubated tend to have one or more of a panic, gag, or suffocation reflex that can result in quick and unexpected patient movements that can adversely affect the desired open configuration of the passage associated with the endotracheal tube assembly. Alteration of the configuration of the tube assembly, and the desired flow of respiration gases associated therewith, can exacerbate the patient panic response. This panic response also commonly includes patient movement of dontia relative to the tube assembly thereby further deviating the intubation tube assembly from a desired position and/or configuration.

For instance, patient movement or repositioning, or repositioning of the exposed end of the endotracheal tube assembly relative to the patient can adversely affect the efficacy associated with utilization of the endotracheal tube assembly. Further, passage of the endotracheal tube assembly through the oral cavity of a patient requires attention to the dontia or jaw movement of the patient. That is, patient repositioning and/or jaw closure or clenching can create a kink, constrict the passage intended to be maintained in an open condition, or even sever the endotracheal tube. Kinking, severing or perforating, or constriction of the endotracheal tube assembly detracts from the ability to achieve the desired patient intubation. Further, repositioning of unconscious patients can create instances of external forces being applied to the head or jaw of a patient in a mouth closing direction which can also adversely affect the desired open configuration of the passage associated with the endotracheal tube assembly. Recognizing such instances, and the dangerous consequences associated with the same, others have attempted in a number of ways to resolve the shortcomings of available appliances for orally incubating patients.

One such example includes a rigid collar or a bite block that is placed about the endotracheal tube and traverses the dontia of the patient. Such appliances complicate the intubation process by requiring placement of the endotracheal tube assembly relative to the patient and subsequent placement of the collar relative to the tube so that the collar traverses the dontia. Further, such a methodology increases the degree that the patient's mouth must be opened to allow the collar to traverse the dontia. Depending on the degree and duration that the patient's mouth is maintained in such an open position can increase patient discomfort during treatment and/or recovery. Further, some bite block assemblies are capable of undesired or unexpected separation of the respective parts of the assemblies. The separated discrete parts are susceptible to being swallowed or translating into the air way of the patients if not timely remediated.

The irregular shape of such accessories can also cause damage to the dontia, such as chipped teeth or the like, should the patient consciously or unconsciously bite down on such accessories. Further, the moveable association between the collar and the endotracheal tube assembly can result in undesirable but unobservable translation of endotracheal tube assembly relative to the collar during use of the same. Accordingly, such a methodology provides a less than a desired solution to resolving the risk of kinking, perforating or severing, and/or compression of the endotracheal tube assembly associated with passage of the endotracheal tube assembly through the oral cavity of the patient.

Still another methodology involves the placement of a strap assembly about the head and/or neck of the patient. The strap assembly is configured to manipulate the position of the mandible to mitigate the ability of the patient to close their jaw while orally intubated. Such methodology suffers from drawbacks similar to those discussed above with respect to associating a collar with a previously placed endotracheal tube assembly.

Still another approach is to provide an armored or reinforced endotracheal tubes that contains a wire that is embedded in the wall of the tube and extends the length of the endotracheal tube in an effort to mitigate kinking. However, the comparative rigidity of such reinforced endotracheal tube assemblies makes it more difficult to use such appliances during intubation such that a stylet or other generally rigid rod, or suitable placement accessory is often utilized to provide a desired contour or shape to the reinforced endotracheal tube during placement.

Further, the metallic coil embedded in the elongated tube, having a generally helical shape, does not mitigate the detrimental effects associated with the patient's biting or otherwise compressing, perforating or severing the endotracheal tube should a patient bite down on the endotracheal tube assembly. There have been reports of intubated patients biting upon the reinforced endotracheal tube assembly and collapsing an at rest shape of the metal reinforcement and the endotracheal tube associated therewith. Still further, other instances have been reported wherein patient bite forces have been sufficient to perforate the endotracheal tube assembly thereby rendering the assembly unusable for suitable or desired intubation. It should be appreciated that the risk associated with a patient biting the endotracheal tube assembly can vary depending upon the nature of the surgery being conducted and that the metal helical reinforcement results in an assembly denoted by a repeating sequence of reinforced/non-reinforced sections of the endotracheal tube assembly that extends along the entire longitudinal length of the assembly making it more difficult to place but still susceptible to failure.

Still further, translating an endotracheal tube assembly relative to an intubated patient via manipulation of the exposed portion of intubation tube assembly can also detract from the efficacy associated with use of the endotracheal tube assembly. On many occasions, an anesthesia provider is asked to move the exposed portions of the endotracheal tube assembly from a right side facing angle relative to the mouth of the patient to a left side facing angle, or vice versa. After such manipulations, the desired bilateral lung function is commonly verified by auscultation or listening to the internal sounds of the body.

Upon removal of many devices manipulated in such a manner, an acute kink can be observed and generally forms approximately 10 cm above the inflatable bladder cuff and in a direction opposite to the natural curvature of the endotracheal tube or toward the side of convexity associated with the hysteresis of the endotracheal tube assembly. Such an indication is indicative of intraoral kinking of the tube or kinking which occurred sometime after tracheal intubation. The shifting of the endotracheal tube from the right hand side to the left hand side of the patient's mouth speculatively led to a torsional force associated with generation of the kink although thermal softening of the tube, due to exposure to body temperature, may have promoted generation of the kink which leads to difficulty in patient ventilation. It has also been observed that such kinking of thermally softened tubes occurs more often upon bending of the in-situ endotracheal tube when manipulated in a convex direction rather than a concave direction relative to an at rest hysteresis of the endotracheal tube assembly.

Therefore, there is a need for an endotracheal tube assembly that can resist kinking, perforation, and/or compression of the endotracheal tube assembly, maintains an open configuration of the endotracheal tube assembly during movement or motion of the patient and for patients in various positions, and does so without unduly interfering with the dontia and/or mandible or increasing patient discomfort associated with being intubated.

### SUMMARY OF THE INVENTION

The present invention discloses an endotracheal tube assembly and method of intubation that resolves one of more of the shortcomings disclosed above. One aspect of the invention discloses an endotracheal tube assembly that is constructed to prevent kinking, perforation, or crushing of the tube due to interaction with the dontia of a patient or a patient position, orientation, or movement. The endotracheal tube assembly includes a tube defined by a hollow core that extends between a first end and a second end of the endotracheal tube assembly. A reinforcement is formed integrally with the endotracheal tube assembly and constructed to prevent constriction of the hollow core by resisting compression of the reinforcement due to patient jaw compression and/or patient position.

Another aspect of the invention discloses an endotracheal tube assembly having a tube that is generally defined by a hollow core that extends between a first end and a second end of the tube assembly. The first end of the tube is constructed to receive a flow from a ventilation appliance and the second end of the tube is configured to pass through the trachea of a patient. A reinforcement is formed integrally with the tube and oriented relative to a longitudinal length of the tube such that the reinforcement traverses dental features of the patient and maintains an open configuration of the hollow core during use of the endotracheal tube assembly.

Another aspect of the invention that is useable with one or more of the above aspects discloses an endotracheal tube assembly having a tube with an elongated shape and that is defined by a hollow core. A first end of the tube remains exposed to atmosphere when a second end of the tube is disposed through the trachea of a patient. An expandable bladder or cuff is fluidly isolated from the hollow core and disposed proximate the second end of the tube. A reinforcement is formed integrally with the tube along the elongated shape nearer the first end than the second end. The reinforcement is inseparable from the tube and has an outer diameter that is no larger than an outer diameter of the tube. The reinforcement is constructed to resist changes to a cross sectional shape of the hollow core, and thereby maintain fluid connectivity between the first end and the second end of the tube via the hollow core, in response to attempted closing of a jaw of the patient. A respiration appliance is connected to the first end of the tube and configured to communicate a respiration material to the second end of the tube to respirate a patient.

Another aspect of the invention that is useable or combinable with one or more of the above embodiments includes a method of orotracheally incubating a patient. The method includes passing an endotracheal tube having a non-compressible section that is formed integrally with the tube through an oral cavity of a patient so that an inflatable balloon cuff that is secured to the tube is disposed proximate a tracheobronchial tree and the non-compressible section of the endotracheal tube extends across dontia of a patient such that the non-compressible section prevents constriction of a hollow core of the endotracheal tube.

These and other features, aspects, and advantages of the present invention will be better understood from the following brief description of the drawings, the drawings, and the detailed description of the preferred embodiment of the present invention.

### DESCRIPTION OF THE DRAWINGS

The drawings illustrate preferred embodiments presently contemplated for carrying out the present invention.

In the drawings:

FIG. 1 is a perspective view of an endotracheal tube assembly according to one embodiment of the invention;

FIG. 2 is a view similar to FIG. 1 of an endotracheal tube assembly according to another embodiment of the invention:

FIG. 3 is a perspective view of a distal or patient facing end of the endotracheal tube assemblies shown in FIGS. 1 and 2; and

FIG. 4 is a graphical anatomical representation of a patient intubated with the endotracheal tube assembly shown in FIG. 1.

### DETAILED DESCRIPTION OF THE PREFERRED EMBODIMENTS

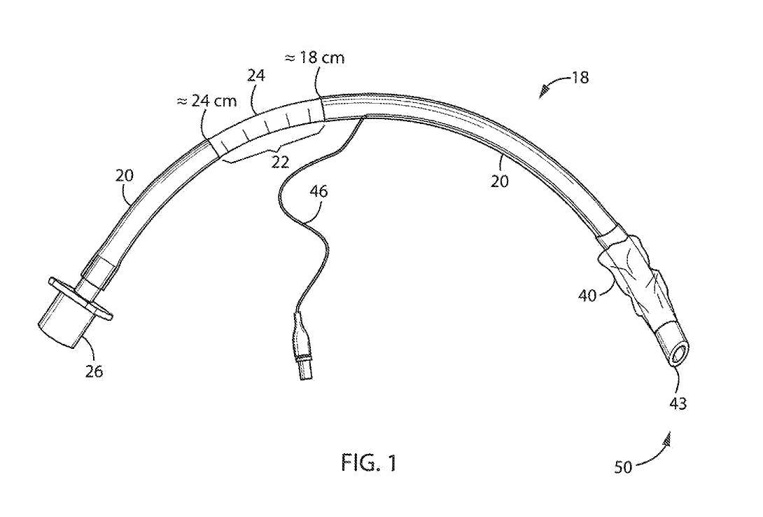

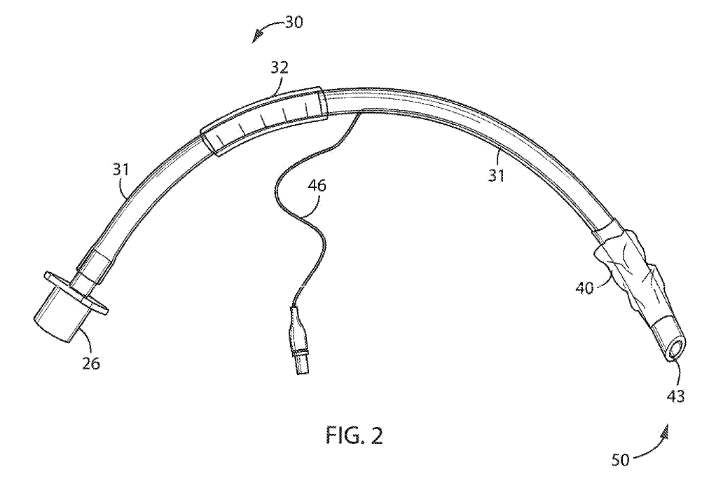

FIGS. 1 and 2 shown endotracheal tube assemblies 18, 30 that each include a respective non-compressible reinforcement 24.32 configured to mitigate incidents of kinking, perforation, or compression due to manipulation of the respective endotracheal tube assemblies 18, 30 relative to the patient, patient movement, and/or interaction with the dontia of the intubated patient as disclosed above. A majority of the longitudinal length of each tube assembly 18, 30 is formed of a flexible plastic tube that is inserted through the mouth of the patient and into the trachea to maintain an unobstructed passageway to deliver oxygen and inhalation anesthetics to the lungs of an intubated patient. The endotracheal tube is commonly placed at a tube depth of approximately 20-23 cm as determined by the upper incisor teeth.

To prevent the patient from compressing the tube by biting on the tube or the tube getting kinked at the teeth, endotracheal tube assembly 18 is formed of a flexible plastic tube portion 20 up to approximately an 18-20 cm from a patient facing end terminus 43 of a respective tube assembly 18, 30. That is the respective reinforcements 24, 32 are formed or otherwise disposed at a location along the longitudinal length of the respective assembly 18, 30 such that the respective reinforcement 24, 32 traverses the dontia of an intubated patient. It is appreciated that other dimensions may be provided to accommodate the anatomy of different patients. In one embodiment, reinforcements 24, 32 are formed along a longitudinal length of the respective tube assembly 18, 30 that is approximately 1.5 cm toward an oral cavity end of the respective assembly 18, 30 from the integration of an another tube, such as a lumen 46, with the respective intubation tube 20. As disclosed further below, lumen 46 is associated with the inflation and deflation of an inflatable bladder or cuff 40 associated with the respective endotracheal tube assembly 18, 30.

Between the approximately 20-24 cm distances of tube 20, 29, the reinforcement 24, 32 of respective endotracheal tube assemblies 18, 30 is formed by, or smoothly transitions to, a shatterproof hard plastic material reinforcement 24, 32 that is more rigid than tube 20 of assemblies 18, 30. Referring to FIG. 1, the internal diameter of tube 20 and reinforcement 24 are generally consistent along the longitudinal length of assembly 18. Preferably, alternate longitudinal ends of reinforcement 24 transition into and out of material or tube 20 to thereby traverse those portions of the assembly 18 that traverse the oral cavity and the thereby the dontia of the patient. Such a construction allows tube 20 to maintain an open internal diameter even when the patient closes their mouth. In a preferred aspect, reinforcement 24 is integrally formed with tube 20 such that an internal diameter of assembly 18 is generally constant along the longitudinal length thereof. It is appreciated that an external diameter of reinforcement 24 can be the same as or less than an external diameter of tube 20. Preferably, the external diameter is reinforcement 24 is no larger than the external diameter of tube 24.

Reinforcements 24, 32 provide a crush and perforation proof portion or assemblies 18, 30 and a reinforcement that has generally uniform mechanical properties about the circumference and longitudinal length associated with the respective reinforcement thereby avoiding the shortcomings of metal coil reinforced tube assemblies as disclosed above. Further, reinforcements 24.32 do not interfere with, detract from, or adversely affect the flexibility or hysteresis associated with the patient facing portion of tube 20 which increases the difficulty associated with the placement of such appliances as also disclosed above.

Reinforcements 24, 32 are selected from materials that mitigate or negate obstruction, compression, peroration or severing of the fluid passage defined by the respective endotracheal tube assembly 18. Reinforcements 24, 32 also prevents kinking or obstruction of the endotracheal tube 20 and assures consistent delivery of oxygen and inhalation anesthetics and exhaling of carbon dioxide during surgical procedures when a patient is any of positioned face down, on his right or left side, oriented head down (Trendelenburg position), or head up (reverse Trendelenburg position). Assemblies 18, 30 maintain a desired respiration path during movement of a patient whether self initiated or if the movement is staff or care provider initiated, executed, or assisted. Assemblies 18, 30 also facilitate more convenient and desired manipulation of the assembly relative to the mouth of the patient without adversely affecting the gas passage associated therewith after patient intubation.

Although material associated with reinforcement 24 preferably extends a distance sufficient to traverse the oral cavity of the patient, it is further envisioned that material of reinforcement 24 can extend to the atmosphere exposed end 26 of assembly 18. Preferably, a connector is provided at end 26 of respective assemblies 18, 30 and is configured to be connected to a respiration appliance, ventilation circuits, or the like, as disclosed further below. As discloses above, it is envisioned that reinforcement 24 can be formed integrally during the construction of tube 20, be formed as a collar or section of shatterproof material internal to tube 20, be external thereto—such as reinforcement 32, or be constructed so as to provide a severable connection between tube 20 and reinforcement 24 at a desired location—such as proximate the 20 cm mark, such that the respective reinforcement 24, 32 extends through the oral cavity and extends to end 26. Preferably, each configuration includes a graduation or other such indicator configured to provide an indication as the insertion depth associated with utilization of respective assemblies 18, 30.

As alluded to above and referring to FIG. 2, endotracheal tube assembly 30 includes a reinforcement structure or reinforcement 32 that is external to the construction of the tube 31. The external orientation of reinforcement 32 renders reinforcement 32 susceptible to translation along tube 31 and presents raised generally orthogonal contours to the external longitudinal profile of tube 31. Although less preferable, it is appreciated that reinforcement 32 associated with assembly 30 could be secured to tube 31 prior to placement of the same and that such a consideration allows assembly 30 to be individualized to particular patient anatomy. Further, reinforcement 32 is generally inseparable from tube 31 during use of assembly 30 thereby mitigating the detriments associated with separation of reinforcement 32 from the assembly 30 during use of the same.

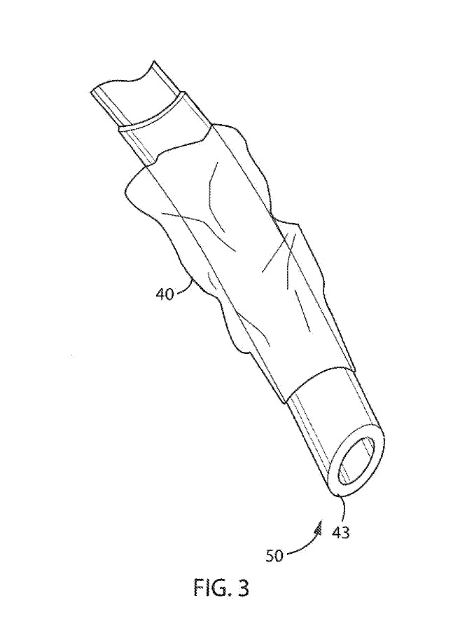

FIG. 3 is a perspective view a patient facing end 50 of assemblies 18, 30. As alluded to above, inflatable balloon, or cuff 40 is commonly associated with a terminus 43 of patient facing end 50 of assemblies 18, 30. As disclosed further below, use of assemblies 18, 30 are configured to be disposed in a patient and accommodate the desired flow of the intubation gases between the lungs of the patient and a discharge end of the respective tube assembly 18, 30.

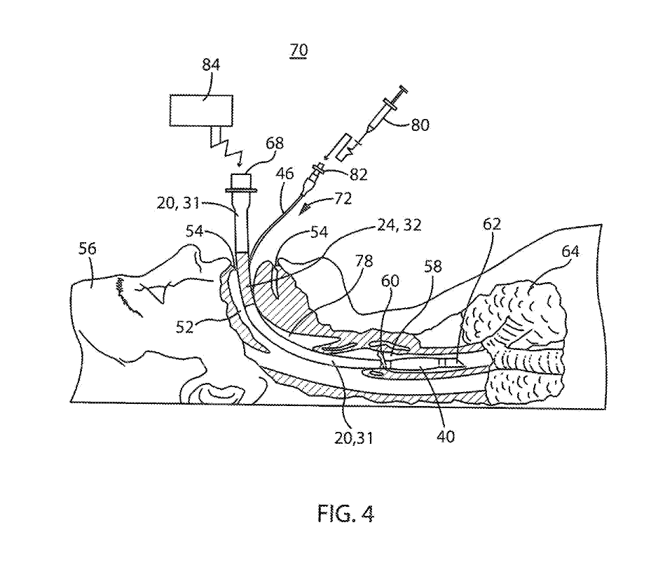

As shown in FIG. 4, during use of assemblies 18, 30, tube portions 20, 31 are passed through an oral cavity 52, and beyond the dontia 54, of a patient 56, such that cuff 40 is disposed toward a lung side 58 of the patient epiglottis 60 and such that a patient end or second end 62 of the respective assembly 18, 30 is disposed proximate the lungs 64 of the patient 56 such that an exposed or first end 68 of assemblies 18, 30 remains exposed to atmosphere 70 proximate the mouth 72 of patient 56. The second tube or lumen 46 is fluidly connected to the volume associated with cuff 40 such that the introduction of a gas or fluid via lumen 46 expands cuff 40 into generally sealed engagement with the inner facing side of the esophagus of patient 56 such that respiration gases are communicated to lungs 64 via the passage defined by tube 20, 31 of assemblies 18, 30.

Reinforcement 24, 32 traverses at least the oral cavity 52 and dontia 54 of patient 56 but does not extend substantially into a throat 78 of patient 56 when the respective assembly 18, 30 is oriented in the desired position relative to epiglottis 60. Reinforcements 24, 32 are generally non-compressible and provide a uniform circumferential rigidity to respective assemblies 18, 30 along the entire longitudinal length of the respective reinforcement 24, 32. It should further be appreciated that reinforcements 24, 32 are constructed to materials selected to be non-crushable and non penetrable by forces associated with human jaw closure pressures such that the internal cross sectional shape associated with the respiration passage defined of the respective tube portions 20, 31 of the respective assembly 18, 30.

A syringe 80 or other such appliance can be utilized to effectuate inflation andfor deflation of cuff 40 during placement and/or extraction of the respective endotracheal tube assembly 18, 30 via operative cooperation of the same with an exposed end 82 associated with the second tube or lumen 46. Upon desired placement of a respective assembly 18, 30, a respiration appliance 84 communicates respiration gases and/or medicinals to lungs 64 of patient 56 via the passage associated with tube portion 20, 31 and/or reinforcement 24 when the interior surface of the restriction is exposed to the passage. It is appreciated that reinforcement 24 could be generally encapsulated by the same material as tube portion 20, 31 such that the reinforcement is isolated from exposure to the materials communicated via the respective tube portion 20, 31.

Removal of respective assemblies 18, 30 is effectuated by deflation of cuff 40 and extraction of the respective assembly 18, 30 via the mouth of patient 56. Reinforcements 24, 32 provide a structure that can withstand patient biting forces thereby mitigating perforation of tube 20, 31. Further terminating reinforcement 24, 32 well short of the patient end 62 of tube 20, 31 or nearer first or atmosphere end 68 of tube 20, 31 provides an endotracheal tube assembly wherein the portion that must traverse the throat 78 of the user is pliable and flexible enough to be easily configured to contour to the anatomy of the patient without undue manipulation of the same and/or use of non-ordinary extraneous guiding tools or the like. That is, user's stilled in the placement of conventional but crushable tube assemblies will be familiar with the placement and use of assemblies 18. 30 as assemblies 18, 30 will provide a tactile responsiveness to which technicians are accustomed. Further, the robust construction of reinforcements 24, 32, allows rotational and translational manipulation of placed assemblies 18, 30 in a manner that limits or wholly mitigates torsional kinking of the more supple or bendable portions of the respective assemblies 18, 30.

Therefore, one embodiment of the present invention includes an endotracheal tube assembly having a tube that is generally defined by a hollow core that extends between a first end and a second end of the tube assembly. The first end of the tube is constructed to receive a flow from a ventilation appliance and the second end is configured to pass through the trachea of a patient. A reinforcement is formed integrally with the tube and oriented relative to a longitudinal length of the tube such that the reinforcement traverses dental features of the patient and maintains an open configuration of the hollow core during use of the endotracheal tube assembly.

Another embodiment of the present invention that is useable with one or more of the features of the above embodiments discloses an endotracheal tube assembly having a tube with an elongated shape and that is defined by a hollow core. A first end of the tube remains exposed to atmosphere when a second end of the tube is disposed through the trachea of a patient. An expandable bladder or cuff is fluidly isolated from the hollow core and disposed proximate the second end of the tube. A reinforcement is formed integrally with the tube along the elongated shape nearer the first end than the second end. The reinforcement is inseparable from the tube and has an outer diameter that is no larger than an outer diameter of the tube. The reinforcement is constructed to resist changes to a cross sectional shape of the hollow core, and thereby maintain fluid connectivity between the first end and the second end of the tube via the hollow core, in response to attempted closing of a jaw of the patient. A respiration appliance is connected to the first end of the tube and configured to communicate a respiration material to the second end of the tube to respirate a patient.

Another embodiment of the invention that is useable or combinable with one or more of the above embodiments includes a method of orotracheally incubating a patient. The method includes passing an endotracheal tube having a non-compressible section that is formed integrally with the tube through an oral cavity of a patient so that an inflatable balloon cuff that is secured to the tube is disposed proximate a tracheobronchial tree and the non-compressible section of the endotracheal tube extends across a dontia of a patient such that the non-compressible section prevents constriction of a hollow core of the endotracheal tube.

The present invention has been described in terms of the preferred embodiments. The embodiments disclosed herein are related as being directed to the assembly as generally shown in the drawings. It is recognized that equivalents, alternatives, and modifications, aside from those expressly stated, the embodiments summarized, or the embodiment shown in the drawings, are possible and within the scope of the appending claims. The appending claims cover all such alternatives and equivalents.

What is claimed is:

1. An endotracheal tube assembly comprising:
a tube having a hollow core and a first end that is constructed to receive a flow from a ventilation appliance and a second end that is configured to pass through the trachea of a patient; and
a reinforcement formed integrally with the tube and oriented relative to a longitudinal length of the tube such that the reinforcement traverses dental features of the patient and maintains an open configuration of the hollow core.

2. The endotracheal tube assembly of claim 1 wherein the tube is further defined by a first compressible portion that extends a longitudinal length of approximately 18 centimeters and the reinforcement extends in the longitudinal direction nearer the first end of the tube.

3. The endotracheal tube assembly of claim 2 wherein the reinforcement has an outer diameter that is no larger than an outer diameter of the first compressible portion.

4. The endotracheal tube assembly of claim 1 wherein the reinforcement is more rigid than the tube.

5. The endotracheal tube assembly of claim 1 further comprising an inflatable cuff proximate the second end of the tube.

6. The endotracheal tube assembly of claim 5 further comprising another tube extending along the tube and fluidly connected to a volume defined by the inflatable cuff.

7. An endotracheal tube assembly system comprising:
a tube having an elongated shape defined by a hollow core and a first end and a second end wherein the first end remains exposed to atmosphere when the second end is disposed through the trachea of a patient;
an expandable bladder fluidly isolated from the hollow core and disposed proximate the second end of the tube;
a reinforcement formed integrally with the tube along the elongated shape nearer the first end than the second end, the reinforcement being inseparable from the tube and having an outer diameter that is no larger than an outer diameter of the tube, the reinforcement constructed to resist changes to a cross sectional shape of the hollow core, and thereby maintain fluid connectivity between the first end and the second end of the tube via the hollow core, in response to attempted closing of a jaw of the patient; and
a respiration appliance connected to the first end of the tube and configured to communicate a respiration material to the second end of the tube.

8. The endotracheal tube assembly of claim 7 further comprising a scale applied to at least one of an exterior surface of the tube and an exterior surface of the reinforcement and providing an indication of a distance between the second end of the tube and respective graduations associated with the scale.

9. The endotracheal tube assembly of claim 7 further comprising another tube that extends along at least a portion of the tube and has a first end that is exposed to atmosphere and a second end that is fluidly connected to a volume associated with the expandable bladder.

10. The endotracheal tube assembly of claim 9 wherein the expandable bladder maintains a position of the tube relative to a patient when the bladder is expanded.

11. The endotracheal tube assembly of claim 9 wherein the another tube is one of formed on an exterior surface of the tube, secured to the tube, formed within a boundary of the tube.

12. The endotracheal tube assembly of claim 11 further comprising an inflator configured to be fluidly connected to the another tube for inflating the bladder and a valve configured to isolate an inflated bladder from atmosphere.

13. The endotracheal tube assembly of claim 9 wherein the tube, reinforcement, and another tube are inseparable without destruction of the endotracheal tube assembly.

14. A method of orotracheally incubating a patient, the method comprising:
passing an endotracheal tube having a non-compressible section that is formed integrally with the tube through an oral cavity of a patient so that an inflatable balloon cuff secured to the tube is disposed proximate a tracheobronchial tree and the non-compressible section of the endotracheal tube extends across a dontia of a patient.

15. The method of claim 14 further comprising inflating the inflatable balloon cuff after placement of the endotracheal tube relative to a patient.

16. The method of claim 15 further comprising deflating the inflatable balloon cuff prior to extraction of the endotracheal tube from a patient.

17. The method of claim 14 further comprising aligning an indicator formed on the endotracheal tube with incisors of the dontia of a patient during placement of the endotracheal tube.

18. The method of claim 17 wherein aligning the indicator with incisors of the dontia further comprises selecting a respective indicator associated with a type of patient from one of a plurality of indicators formed along at least a portion of a longitudinal length of the endotracheal tube.

19. The method of claim 14 further comprising communicating respiration gases through a hollow core of the endotracheal tube.

20. The method of claim 19 further comprising communicating a treatment drug through the hollow core of the endotracheal tube to a patient.
